(19) United States
(12) Patent Application Publication
(10) Pub. No.: US 2007/0014729 A1
(43) Pub. Date: Jan. 18, 2007

(54) Tissue engineered scaffolds and mehtods of preparation thereof

(75) Inventors: Walid A. Farhat, Toronto (CA); Herman Yeger, Richmond Hill (CA)

Correspondence Address: BERESKIN AND PARR, 40 KING STREET WEST, BOX 401, TORONTO, ON, M5H 3Y2, CA

(73) Assignee: The Hospital for Sick Children, Toronto (CA)

(21) Appl. No.: 11/377,571

(22) Filed: Mar. 17, 2006

Related U.S. Application Data
(60) Provisional application No. 60/688,689, filed on Jun. 9, 2005.

(51) Int. Cl. A61K 35/37

(52) U.S. Cl. 424/9.34; 424/426; 424/550

(57) ABSTRACT

There is provided scaffolds for tissue repair/augmentation/implant comprising an acellular matrix, a biocompatible polymer and a biomimetic agent. The scaffolds advantageously supports cell growth in the target tissue. There is also provided a method for the preparation of the scaffold and for monitoring the functionality of the scaffold in tissue using dynamic contrast-enhanced magnetic resonance imaging.

### CROSS-REFERENCE TO RELATED APPLICATION

This application claim priority on U.S. provisional application No. 60/688,689 entitled “Tissue engineered scaffolds for incorporation of biomimetics molecules and methods of preparation thereof” filed Jun. 9, 2005.

### FIELD OF THE INVENTION

The invention relates to the field of tissue grafts and more specifically to tissue engineered scaffolds and preparation thereof for implantation.

### BACKGROUND OF THE INVENTION

Tissue and organ transplantation is a rapidly growing therapeutic field as a result of improvements in surgical procedures, advancements in immunosuppressive drugs and increased knowledge of graft/host interaction. However, tissue transplantation remains associated with complications including inflammation, degradation, scarring, contracture, calcification (hardening), occlusion and rejection. There are numerous investigations underway directed toward the engineering of improved transplantable tissue grafts, however, it is generally believed that ideal implants have yet to be produced.

Autologous or self-derived human tissue is often used for transplant procedures. The motive for using autologous tissue for transplantation is based upon the concept that complications of immunorejection will be eliminated, resulting in enhanced conditions for graft survival.

When the supply of transplantable autologous tissues is depleted, or when there is no suitable autologous tissue available for transplant, then substitutes may be used, including man-made synthetic materials, animal-derived tissues and tissue products, or allogeneic human tissues donated from another individual. Man-made implant materials include synthetic polymers (e.g. (PTFE) polytetrafluroethylene, Dacron and Goretex). Additionally, man-made synthetics (polyurethanes) and hydrocolloids or gels may be used as temporary wound dressings prior to split-skin grafting.

Despite these advances the main challenge is to create and maintain a functionally and biologically compatible tissue-engineered construct. For example new vessel formation is essential for ensuring a sufficient supply of oxygen and nutrients to sustain cell survival and promote proliferation in the reconstructed tissue (Zisch et al. Cardiovasc Pathol. 2003 12:295). In fact, for large organs such as the bladder, achieving an immediately perfused vascular tree is crucial in the initial phase after implantation (Nor et al. Lab Invest 2001, 81:453).

Thus improved transplantable tissue-engineered scaffolds are needed.

### SUMMARY OF THE INVENTION

There is provided tissue engineered scaffolds for organ replacement/repair/augmentation and methods for making same.

In one embodiment there is provided a scaffold for implant in a mammal comprising an acellular matrix, one or more biocompatible polymer and one or more biomimetic molecule such as an angiogenic agent.

In another embodiment there is provided a method for preparing an acellular matrix comprising: obtaining a tissue sample from a biological tissue; treating the tissue to generate a substantially intact extracellular matrix with the proviso that the step of extracting does not comprise treatment of the tissue sample with an anionic detergent

In yet another embodiment there is provided a method for monitoring tissue functionality in an individual the method comprising, injecting a nuclear magnetic resonance contrast agent in the individual, obtaining DCE-MRI measurements in a region of interest (ROI) of the tissue, and correlating the measurements with a parameter indicative of functionality.

There is also provided a method for treating an individual in need of organ replacement/augmentation comprising: determining a need for the individual for an implant, implanting a scaffold as described above and monitoring the implant over time for assessing the organ functionality.

### BRIEF DESCRIPTION OF THE DRAWINGS

Further features and advantages of the present invention will become apparent from the following detailed description, taken in combination with the appended drawings, in which:

FIG. 1 is a schematic diagram of the apparatus used for measuring porosity;

FIG. 2 is a porosity index bar graph;

FIG. 3 is a graphic of water porosity for ACMs as a function of time;

FIG. 4 are graphics of tensile strength of fresh porcine bladder and ACM preparations;

FIG. 5A is a photomicrograph of ACM on which urothelial and smooth muscle cells were seeded;

FIG. 5B is a photomicrograph of ACM on which urothelial and smooth muscle cells were seeded by spraying and to which fibrin glue was added;

FIG. 6A shows photomicrographs of ACM with and without HA using alcian blue staining;

FIG. 6B shows photomicrographs of ACM with and without HA using H&E staining;

FIGS. 7A and B show routine H&E sections of urinary bladder (A, control) and detergent-extracted urinary bladder ACM (B, ACM) confirm the absence of cells (urothelial, smooth muscle, or endothelial cells);

FIG. 7C using a sensitive fluorescence-based quantification method, the entire DNA content of fresh and processed tissues were measured to confirm absence of DNA in the ACM. 1, DNA 6 g (2 g/mL): control positive; 2, porcine ACM DNA 5 L; 3, porcine ACM DNA 15 L; 4, blank: control negative; 5, normal porcine bladder DNA 5 L; 6, normal porcine bladder DNA 15 L;

FIG. 8a shows Alcian's blue stain: ACM with no HA (control);

FIG. 8b shows HA incorporation into the ACM: blue stain indicates HA;

FIG. 8c shows HA retention into the ACM after subjecting the ACM/HA to the porosity testing;

FIG. 9A shows box plots of the porosity indices (cc/cm² hr) for the three groups. (L-ACM, lyophilized/rehydrated ACM; HA-ACM; ACM, untreated ACM);

FIG. 9B shows data summary of the porosity indices (cc/cm² hr) for the three groups. (L-ACM, lyophilized/rehydrated ACM; HA-ACM; ACM, untreated ACM);

FIG. 10 shows Rabbit bladder grafts (arrows) imaged 1 week post-implantation. a) Pre-injection sagittal, fat-suppressed T1-weighted MRI. b) T1-weighted difference MRI 4-minutes postinjection. VEGF dosage used in graft preparation: 0 ng/g (control), 10 ng/g (low VEGF), 20 ng/g (high VEGF);

FIG. 11 are Concentration-time curves in various preparations of bladder grafts at 1 week postimplantation. VEGF dosage used in graft preparation: 0 ng/g (control), 10 ng/g (low VEGF), 20 ng/g (high VEGF). Data represents mean±SE in uniformly enhancing ROIs taken across four to five contiguous 3-mm thick imaging slices;

FIG. 12 shows that DCE-MRI parameter K trans (mean±SE) provides discrimination of the control and low VEGF groups but not between the low and high VEGF groups within 2 weeks post-implantation;

FIG. 13 shows that DCE-MRI parameter AUC8 min (mean±SE) discriminates amongst all VEGF groups;

FIG. 14 shows composite images of immunostained sections showing microvessels in various preparations of bladder grafts at 1 week post-implantation;

FIG. 25 shows a comparison of CD31 microvessel density (MVD) and DCE-MRI in different VEGF groups. Values shown are mean±SD normalized to the mean value of the control group;

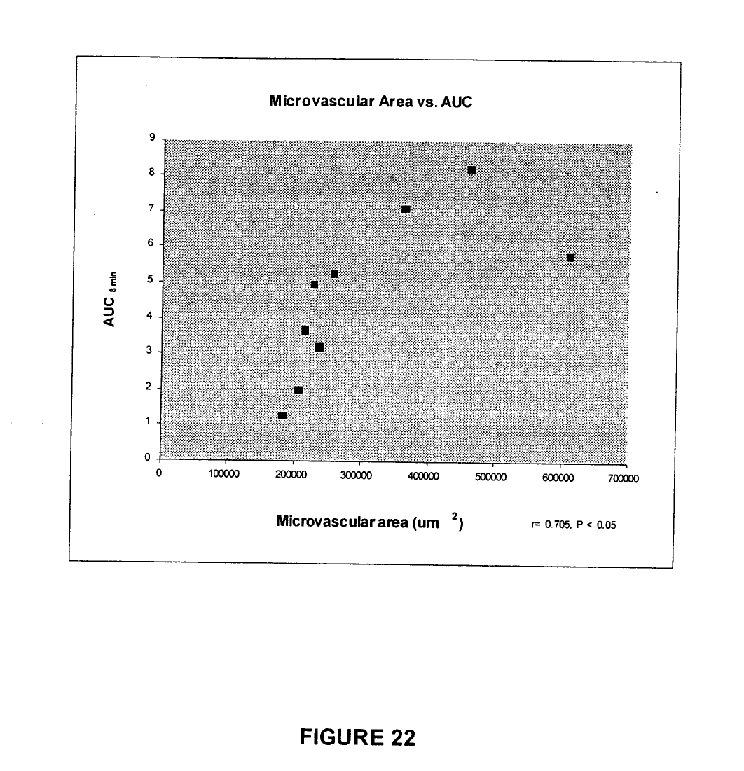

FIG. 16 shows DCE-MRI parameter K trans versus microvessel density (MVD). The correlation is not significant, especially amongst the low and high VEGF groups (r=0.572, P>0.10);

FIG. 17 shows DCE-MRI parameter AUC8 min versus microvessel density (MVD). The correlation is significant but is not strictly linear (r=0.705, P<0.05);

FIG. 18A shows composite images of CD-31 immunostained grafts at 3 weeks for the three VEGF concentrations demonstrate markedly increased microvascular area with high VEGF concentration;

FIG. 18B shows H&E staining at three weeks shows improved cellularity and decreased fibrosis in the high VEGF group when compared to the control and low VEGF group;

FIG. 18C shows a Masson trichrome staining at three weeks shows improved cellularity and decreased fibrosis in the high VEGF group when compared to the control and low VEGF group;

FIG. 19 shows microvascular area over time for varying VEGF concentrations;

FIG. 20 shows the post-contrast T1-weighted difference MRI identifies ACM grafts on the anterior wall of the bladder (arrow);

FIG. 21 shows the DCE-MRI area under the concentration-time curve for contrast uptake by the grafts at 8 minutes for varying VEGF concentrations over different implantation times; and

FIG. 22 shows that DCE-MRI parameter AUC_{8 min} correlates with microvascular area and correctly ranks the data.

### DETAILED DESCRIPTION OF THE INVENTION

The present invention relates to scaffolds as implants for organ repair/replacement/augmentation. In one embodiment the scaffold comprises an acellular matrix (ACM) capable of supporting cell growth and vascularization/angiogenesis. The ACM can be chemically synthesized or can be derived from existing tissue by obtaining and treating the tissue as will be described below.

In the present invention it was advantageously found that by omitting the extraction of the tissue sample with an anionic detergent it is possible to obtain an ACM with better overall properties for organ replacement/repair/augmentation. For example, the ACM obtained by the method of the present invention allowed better cell growth in the matrix and also surprisingly resulted in better retention of the proteins in the ACM as indicated in table 1 which presents the composition of an ACM obtained from porcine bladder. Thus, in one embodiment of the present invention there is provided a novel method for the preparation of an ACM which comprises: obtaining a tissue sample, which may be isolated from a suitable donor, and extracting the tissue sample to produce a matrix comprising essentially extracellular matrix proteins without using anionic detergent. In one embodiment the extraction, which is mainly for the purpose of removing cellular material, is achieved by lysing the cells present in the tissue, degrading the nucleic acids and extracting the tissue so as to remove the cellular components/debris. In a preferred embodiment, the method for ACM preparation is generally as described in U.S. Pat. No. 4,776,853, which is incorporated herein by reference. The method comprises treating the tissue sample with a hypotonic buffer solution at a mild alkaline pH for rupturing cells of the tissue sample, the hypotonic buffer solution including active amounts of proteolytic inhibitors and preferably active amounts of antibiotic, extracting the tissue sample with a buffered solution having a high concentration of salt, the solution being at a mild alkaline pH, subjecting the tissue sample to enzymatic digestion in a buffered saline solution, the enzymes consisting of purified protease-free deoxyribonuclease and ribonuclease, again extracting the tissue with a buffered solution at mild alkaline pH containing at least one non-ionic detergent and preferably two non-ionic detergents and active amounts of antibiotics and optionally storing the processed tissue sample in physiologic saline.

The extractions are preferably performed at 4° C. while stirring the solution for a sufficient time, preferably about 24 hours. The high salt extraction is followed by a balanced salt solution wash preferably at room temperature for a sufficient amount of time, preferably about 1 hour. The enzymatic digestion is performed at a temperature compatible with enzyme activation and while any temperature within a range of temperatures that would be known to one skilled in the art is appropriate, a preferred temperature is about 37° C. At this temperature the digestion is preferably carried on for approximately 6 hours but it will be appreciated by those skilled in the art that the incubation time will vary according to the temperature. The final extraction procedure is preferably followed by one or more washes in sterile distilled water at 4° C. Of course the temperatures and times stated above may be modified within ranges that can be determined by those skilled in the art based on the particular circumstances (such as type of enzyme used, quantity of material etc.).

TABLE 1

| | | Acellular | Acellular |
|---|---|---|---|
| | | Matrix SDS | Matrix SDS |
| Stain | Normal Bladder | free (ACM) | based (ACM) |
| Laminin | 4 | 3 | 2 |
| Fibronectin | 4 | 2 | 2 |
| Cytokeratin 7 | 4 | 0 | 0 |
| Collagen IV | 4 | 3 | 2 |
| Collagen I | 4 | 4 | 4 |
| Collagen III | 4 | 4 | 4 |
| Desmin | 4 | 2 | 1 |
| SM Actin | 4 | 3 | 3 |
| SM Myosin | 4 | 3 | 3 |
| Vimentin | 4 | 3 | 1 |
| PAN Neurofilament | 4 | 3 | 1 |

Semiquantitative summary of immunohistochemical staining in normal bladder and ACM Scoring was done on a scale of 0 to 4 where ‘0’ represents no staining and ‘4’ represents maximum staining as seen in normal bladder.

The ACM may be obtained from the same kind of tissue as the target tissue or organ. However, heterologous tissue may also be suitable. For example, tissue from tendons has been shown to be adequate for bladder augmentation.

The ACM is treated to make it biocompatible with and functional for the target tissue/organ. By biocompatible it is meant that the properties of the matrix should be such as to permit the implant to achieve some or all of the functions normally carried out by the tissue.

Thus the ACM is preferably treated to allow cell attachment and growth to repopulate the matrix with cells of the same type (autologous cells) as that of the target organ/tissue. Preferably the cells originate from the tissue or organ that is to be augmented or replaced or repaired (autologous cells).

In a preferred embodiment the matrix is treated with a polysaccharide such as a glycan for example an glycosaminoglycan to improve cellular attachment and growth. The polysaccharide may be incorporated or associated with the matrix using a freeze-dried process. Thus the ACM can be freeze-dried and rehydrated with a polysaccharide solution over a period of time. The amount of polysaccharide(s) into the ACM may depend on the molecular weight of the polysaccharide(s). In general, there is an inverse relationship between the polysaccharides molecular weight and optimal polysaccharides concentration for optimal incorporation into the ACM. Thus high molecular weight polysaccharides exhibit a higher incorporation ratio when incubated with the ACM at low concentrations. Incorporation may be effected in a stepwise (sequential) fashion by which the ACM is sequentially incubated with a series of polysaccharide(s) solutions. The method of the present invention has been successful in incorporating polyssacharide in ACM of relatively large dimensions. In a preferred embodiment the molecular weight of polysaccharides is between 62,000 and 99,000,000.

The choice of the polysaccharide to be incorporated in the ACM depends on the properties of the target tissue or organ. For example, it has been found that hyaluronic acid is suitable for bladder tissue. Without wishing to be bound by any theory, the negative charge of HA and its hygroscopic nature may contribute to its water retention (or barrier) capability. Thus it will be appreciated that the physico-chemical properties of the polyssacharide will play a role in its suitability for incorporation in an ACM destined to be implanted in a particular tissue or organ. The nature of the testing to be done on an ACM comprising one or more polysaccharides to assess its suitability, depends on the target tissue/organ and can be determined by those skilled in the art. It will be appreciated that biocompatible polymers including polymers such as non-biological (synthetic) polymer, polysaccharide-like polymers and the like may also be used in association with the ACM. By biocompatible polymers it is meant polymers that can be associated with the matrix and that possess mechanical and physiological properties compatible with the target tissue. An example of a property is the ability to support cell growth.

Furthermore, it has been found that polysaccharide-containing ACM provides a suitable environment for incorporation of biomimetic agents. By biomimetic agents it is meant any molecule capable of regulating a physiological response. non-limiting examples include: hormones, growth factors, angiogenic agents, nerve growth factors, bone growth factors and the like. For example, angiogenic growth factors can be incorporated in the matrix to promote the development of blood vessels on the implants to sustain cell growth by increasing blood flow to the implant. VEGF has been shown to promote angiogenesis within a scaffold as will be described below. Furthermore, VEGF has also been shown to reduce inflammatory response in the implant thereby reducing graft/implant fibrosis. It will be appreciated that other angiogenic factors may also be employed either alone or in combination with other angiogenic or biomimetic agents such as hormones. The choice of biomimetic agents may depend on the type of organ or tissue in which the scaffold is implanted and the desired physiological properties.

As mentioned above, the composition of the scaffold will be optimized for the physiological properties required in each particular case. It is therefore desirable to assess the functional reconstitution of the scaffold before and after implantation in the organ. Different aspects may be evaluated such as the mechanical properties, patency, physiological response to growth factors and the like using known techniques such as imaging techniques (ultrasound, x-ray and the like) and physiological assays.

In this respect, angiogenesis can be an important aspect to evaluate for assessing the efficiency of neo-vascularization of the graft/implant but it is a difficult assessment to perform. Conventional assessment using histology and microscopy is not useful for in vivo assessment and is limited in accuracy by failing, for example, to provide 3 dimensional information. Thus in one embodiment of the invention there is provided a method for assessing angiogenesis in grafts/implants using dynamic contrast-enhanced magnetic resonance imaging (DCE-MRI). It has been advantageously discovered that DCE-MRI measurements can be correlated with functionality of tissue-engineered grafts/implants.

The method comprises the acquisition of MRI images to identify the region of interest (ROI) comprising the graft/implant. For example ROI can be localized using T₂-weighted multislice fast spin-echo (FSE) image acquisition. Functional images, that is to say images that can be used to assess functional aspects of the graft/implant, may be obtained by injecting a contrast agent and acquiring dynamic images. In a preferred embodiment dynamic images are acquired using 3D T₁-weighted fast spoiled gradient recalled echo (SPGR) sequence. The dynamic images are preferably acquired prior to, during and for a period of time following the injection of the contrast agent. The period of time during which images will be acquired after injection of the contrast agent will depend on the pharmacokinetic of the contrast agent and the type of measurement being performed. Contrast agents are well known to those skilled in the art and may include but are not limited to Gd-DTPA.

The dynamically acquired images provide data that can be used to estimate functional parameters. For example, the concentration of contrast agent within a ROI can be monitored over time and used to derive functional parameters such as endothelial transfer constant (K^{trans}), fractional plasma volume and the like. These parameters can be derived based on models including but not limited to enhancement rate (SSₘₐₓ), Tofts and Kermode Model (K^{trans}, νₑ, νₚ) and Uptake integral approach (area-under-the-concentration-time curve AUC). The parameters can be correlated with and therefore serve as a basis for measurement of grafts/implant characteristics that are important for assessing the success of the implantation. In one embodiment it has been found that AUC can be correlated with MVD. Preferably AUC is derived from integration over long period of time.

In one advantageous realization of the present invention the scaffold can be used to treat a patient in need of organ replacement, repair or augmentation. While the present specification uses bladder augmentation as an example, other organs may also be replaced/repaired/augmented using the method described herein. In one embodiment the scaffold may be prepared from pig bladder tissue as described below. It will be appreciated however, that the tissue may be obtained from human tissue banks to provide a homograft that can be assessed for histocompatibility. Method for surgical implantation is known to those skilled in the general art of tissue/organ grafting, transplantation, implantation and augmentation.

The need for a patient to be implanted with a scaffold can be evaluated by accepted diagnosis methods. For example, the need for bladder augmentation/replacement can be assessed by measuring urinary functions such as urine pH, electrolyte composition and the like as well as knowledge of prior treatments affecting the organ such as radiotherapy for cancer treatment. Identification of the need for organ repair/replacement in general is known in the art.

### EXAMPLE 1

### ACM Preparation

Fresh bladder are provided and washed in sterile phosphate buffer saline (PBS). The bladder tissue is then extracted with solution A consisting of 10 mM Tris HCl pH 8.0, 5 mM EDTA, 1% Triton X-100, Petabloc Plus™ (protease inhibitor) 0.1 mg/ml and Antibiotics/Antimycotic. The extraction is performed at 4° C. for 24 hrs with stirring.

The bladder tissue is then extracted with solution B consisting of 10 mM tris HCl pH8.0, 5 mM EDTA, 1% Triton X-100 and 1.5M KCl. The extraction is performed at 4° C. for 24 hrs with stirring.

The second extraction is followed by two washes in Hanks' Balanced Salt soluiton for 1 h each at room temperature and the tissue is then enzymatically digested with DNAse/RNAse solution at 37° C. with shaking for 6 hrs.

A third extraction is performed using solution C consisting of 50 mM Tris HCl pH 8.0, 0.25% CHAPS, 1% Triton X-100, and Antibiotics/Antimycotic with shaking at 4° C. for 24 hrs.

The resulting ACM is then washed 4 times in sterile dH₂O at 4° C. Before being stored in physiological saline.

### EXAMPLE 2

### ACM Characterization

The integrity of the ACM resulting from the ACM preparation method described above was evaluated by studying the spatial distribution of different extracellular matrix components in comparison to normal bladder. Histochemical and immunohistochemical analyses of the modified ACM (M-ACM) showed that the glycosaminoglycans (GAGs) were absent but the contents of collagen I, III, IV (collagen), elastin, laminin, and fibronectin were preserved in a similar spatial topographic distribution to normal bladder (Table 1 supra). This result indicated that the retained components were appropriately located for cell attachment and proliferation.

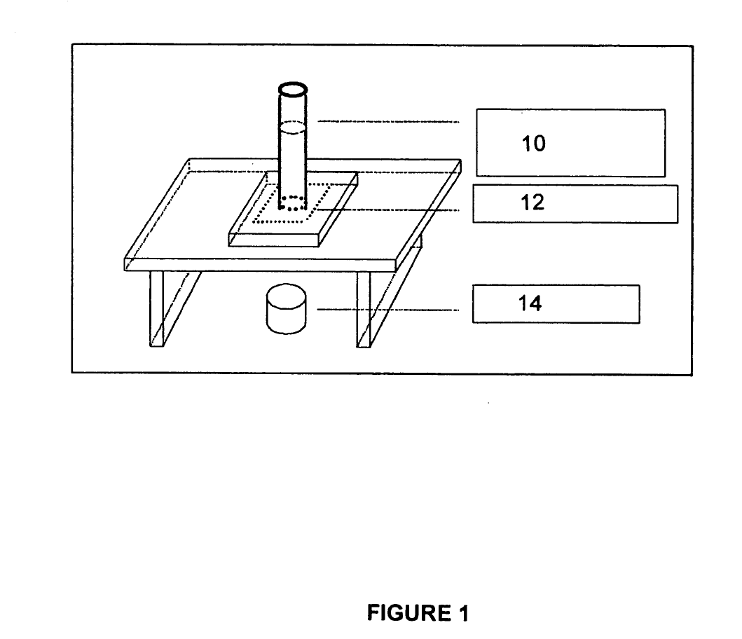

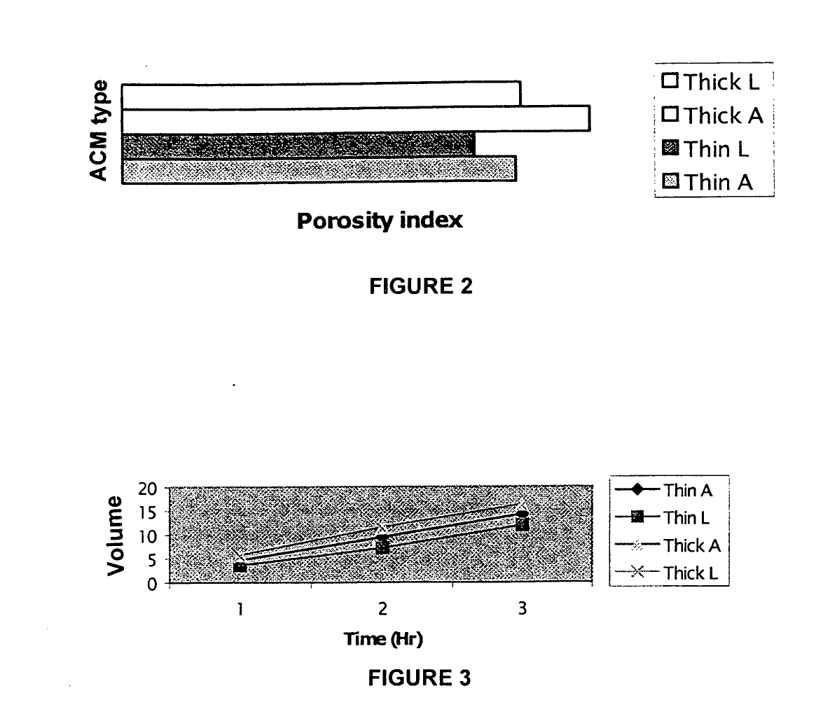

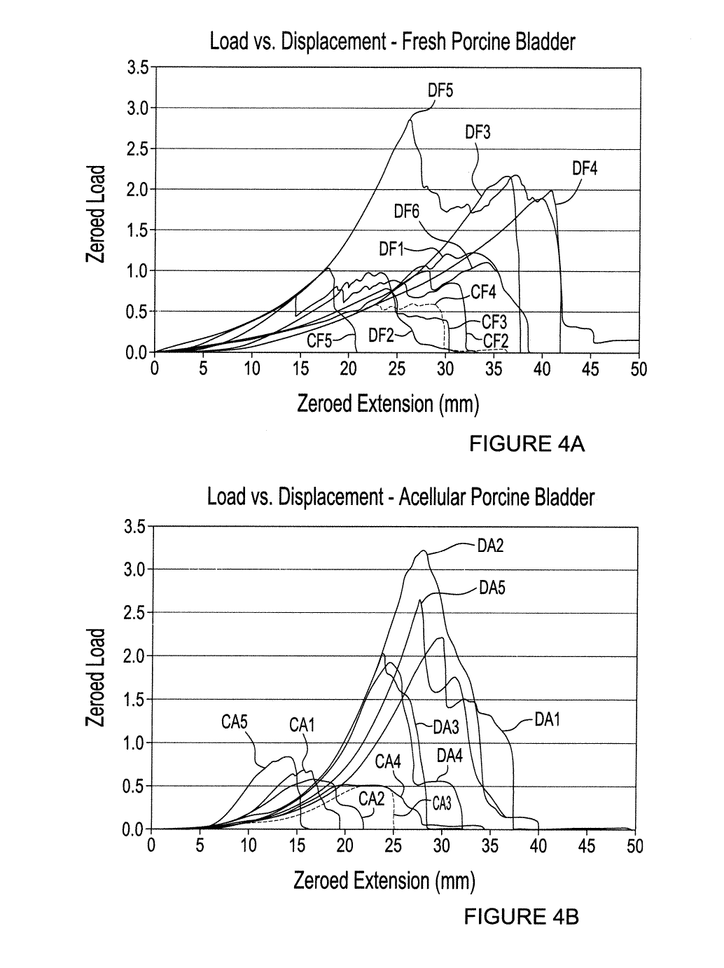

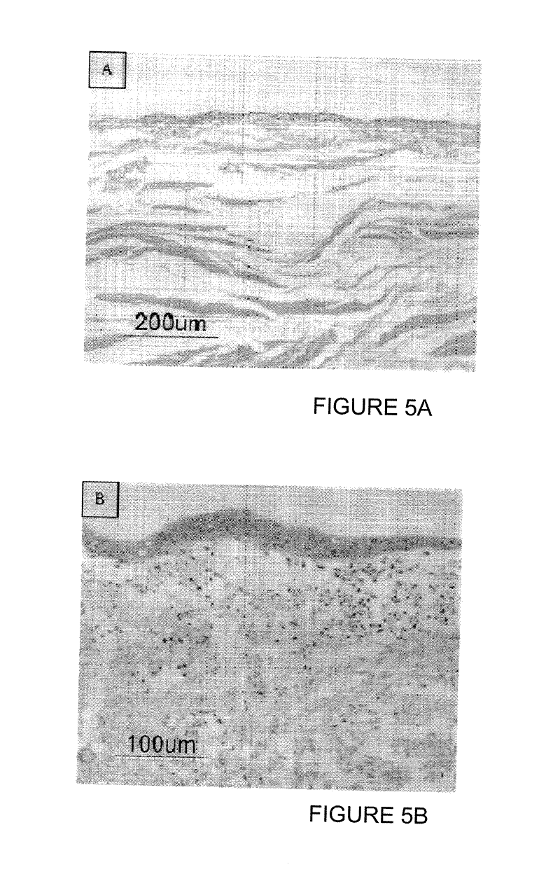

M-ACM Porosity: The apparatus to measure porosity of the bladder tissue is generally described in FIG. 1 in which there is shown a liquid pressure column 10, placed on top of a tissue holder 12 on which the tissue is placed and reservoir 14 to measure liquid volume going through the tissue. Since bladder permeability barrier plays a role in maintaining normal homeostasis the permeability/ porosity of the porcine bladder M-ACM was evaluated. Luminal (L) and abluminal (A) M-ACM specimens were subjected to fixed static DI water pressure (10-cm), and water passing through the specimens was collected at specific time interval (FIGS. 2 and 3). The tensile strength of fresh bladder tissue and ACM was compared and as can be appreciated from FIG. 4 in which the results for 10 samples are shown, the strength is generally preserved in ACM preparations.

In vitro Biocompatibility: For the purpose of autologous cell transplantation on biodegradable scaffolds as an alternative treatment to urinary bladder substitution, we evaluated the biocompatibility of the acellular matrix with bladder cells/matrix interaction in vitro. We first isolated urothelial and smooth muscle cells from porcine urinary bladders using established methods (Baskin et al. J Urol. 1993, 149(1):190; Ludwikowski et al BJU Int. 1999, 84(4):507), and assessed biocompatibility using 2 methods:

A. The direct contact method; the purpose of this experiment was to evaluate if the M-ACM contained residual detergents that might be slowly released which would inhibit cellular growth around and onto the matrix. The M-ACM (luminal and adluminal surfaces) pieces (1 cm²) were placed in the centre of subconfluent monolayers of (porcine urothelial cells) PUC and (smooth muscle cells) SMC.

B. The direct seeding method; the purpose of this experiment was to evaluate if the M-ACM is a good substratum for cell attachment and proliferation. PUC and SMC were cultured on either luminal or abluminal ACM pieces respectively at a density of 1×10⁵ cells per cm².

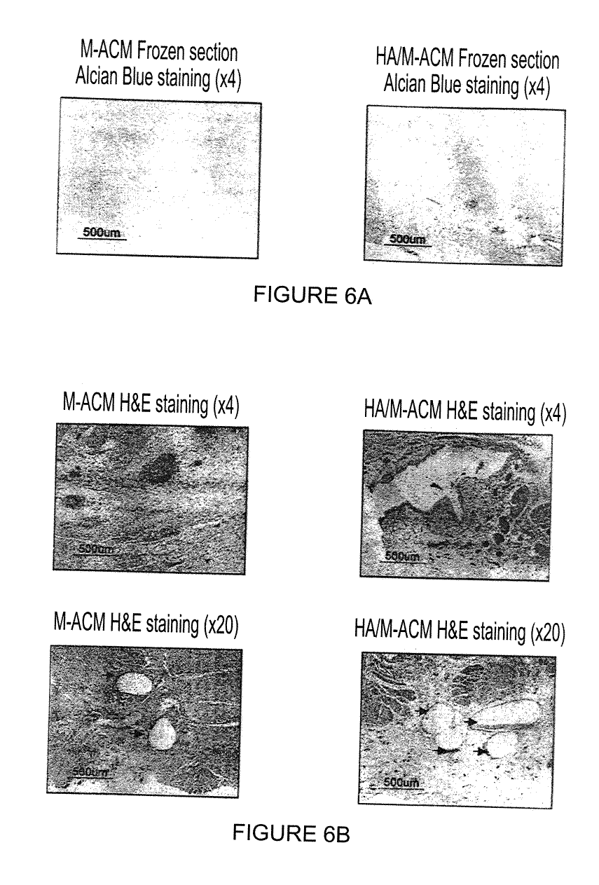

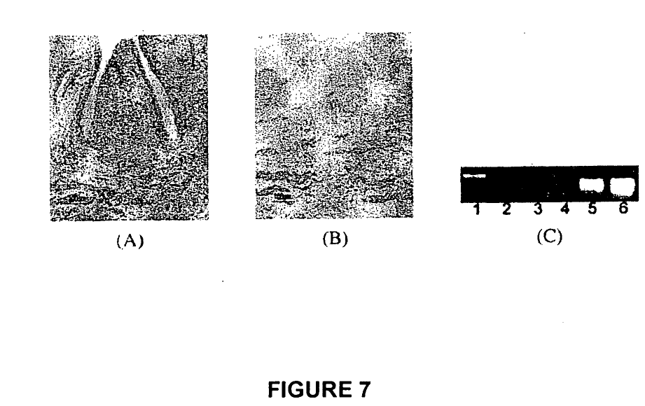

It was evident from these studies that the matrix provided an appropriate environment for cell attachment, survival and growth (FIG. 5 A, B).

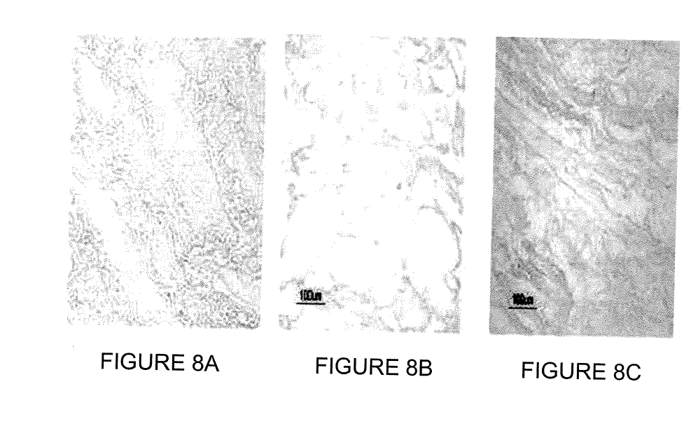

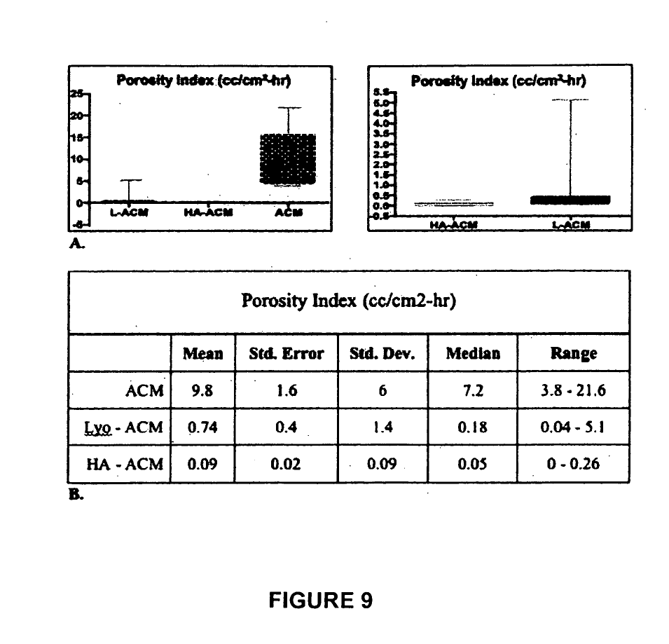

In preliminary studies, using sequential M-ACM lyophylization and then rehydration in hyaluronic acid (HA) as the heteropolysaccharide containing solutions, high molecular weight HA was successfully incorporated into the M-ACM and it was determined that the spatial architectural distribution of the residual extracellular matrix components are maintained. The presence of HA in the M-ACM was confirmed using alcian blue staining (FIG. 6A), and importantly it was demonstrated that HA/M-ACM is non-porous to fluid. In addition, in pilot implantation experiments, examination of HA/M-ACM 40 days post implantation showed a marked decrease in inflammation when compared to M-ACM alone (FIG. 6B). HA used was hyaluronic acid sodium salt from rooster comb [Molecular weight: (1˜4 million.), Sigma-Aldrich Co] but it will be appreciated that other sources of HA (such as rooster comb, bovine vitreous humor or human umbilical cord) or other polysaccharides may be used.

Materials and Methods

Porcine Urinary Bladder Acellularization

Whole porcine urinary bladders were harvested from 20-28 kg pigs at the time of animal killing for other unrelated experimental protocols. They were then acellularized through a proprietary method. Fresh bladders were washed in sterile phosphate buffer saline (PBS) and then stirred in a hypotonic solution of 10 mM Tris HCl, pH 8.0, 5 mM EDTA, 1% Triton X-100, Petabloc Plus™ (protease inhibitor) 0.1 mg/mL, and antibiotics/antimycotic at 4° C. for 24 h to lyse all cellular components. On the second day, the tissue was placed in a hypertonic solution containing 10 mM Tris HCl, pH 8.0, 5 mM EDTA, 1% Triton X-100, and 1.5M KCl and stirred for 24 h at 4° C. to denature residual proteins. Tissue was then washed in Hanks' Balanced Salt solution for 1 h at room temperature twice prior to a 6 h enzymatic digestion with DNAse/RNAse solution at 37° C. with shaking. A final 24 h extraction was performed at 4° C. in 50 mM Tris HCl, pH 8.0, 0.25% CHAPS, 1% Triton X-100, and antibiotics/antimycotic with shaking. The resulting ACM was finally washed four times in sterile dH₂O at 4° C. and then stored in physiological saline.

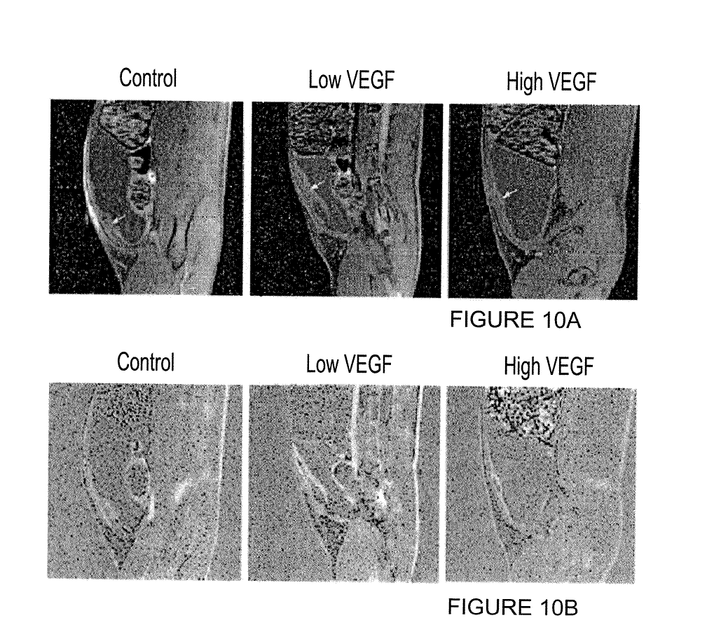

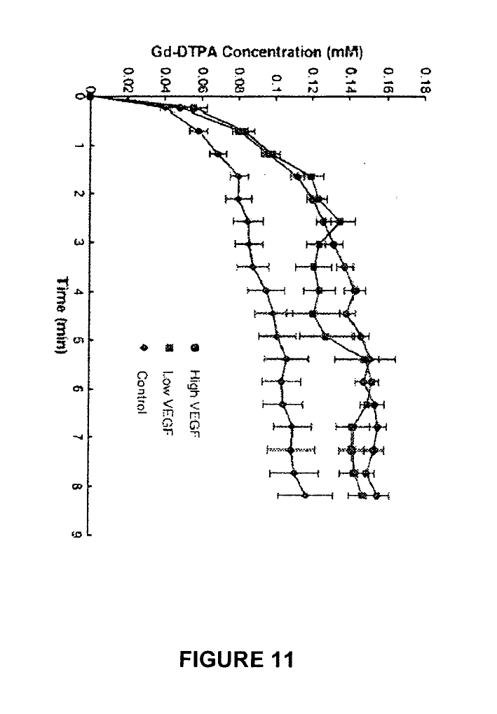

Tissues from the ACM and normal porcine urinary bladder were taken for DNA content, histology, and immunohistochemistry for cellular and extracellular content to confirm the acellular status of the scaffold. H&E (FIG. 7A,B) showed complete acellularization and no detectable urothelium, smooth muscle, or endothelial cells. This was also confirmed by cell type special markers using immunohistochemistry. DNA from fresh and ACM tissues was isolated and DNA-sensitive fluorescent dye assay [DNeasy Tissue Kit (Qiagen Inc., Mississauga, Canada)] was performed (t-test, p<0.001) (FIG. 7C). In addition, histochemical and immunohistochemical analyses of the extracellular matrix components in the modified ACM showed that the glycosaminoglycans (GAGs) were absent, but the contents of collagen I, III, IV (collagen), elastin, laminin, and fibronectin were preserved in a similar spatial topographic distribution to normal bladder.

All experimental protocols were approved by the Animal Care Committee of the Hospital for Sick Children and were in compliance with the Canadian Council for Animal Care guidelines.

Hyaluronic Acid Incorporation

ACM was lyophilized (VirTis-temp, −70° C. and vacuum, 121 millitorr) and then rehydrated over 24 h at 37° C. in increasing concentrations of HA (0.05, 0.1, 0.2, and finally 0.5 mg/100 mL; Sigma, product No.: H5388, USA). HA incorporation was confirmed qualitatively with Alcian blue staining and quantitatively with flourophore-assisted chromatography electrophoresis (FACE).

Porosity Testing

Three groups (n=15/group) of ACM samples were subjected to porosity testing with a device designed at the Hospital for Sick Children (FIG. 1). Of note, the urinary bladder is not normally subjected to a pressure above 10 cm of water. Hence, this device was designed primarily for assessment of porosity in biomaterials intended for urinary bladder substitution. Groups consisted of standard porcine ACM, lyophilized ACM that was rehydrated in distilled water for 24 h at 37° C., and HA-ACM. Specimens were cut into pieces slightly larger than 2×2 cm₂, clamped across the 2-cm diameter aperture of the porosity testing apparatus, and subjected to a static 10 cm H₂O pressure by manually maintaining a 10 cm column of distilled water over the luminal surface of the ACM. Effluent from the ACM was collected and measured hourly for 3 h. Specimen porosity was calculated as the measured effluent volume per unit area over time [porosity=V/At=V(cc effluent in 3 h)/aperture area(cm₂) t(3 hr)=V/9.42 (cc/cm₂ hr)]. After porosity testing, Alcian blue staining was repeated on HA-ACM constructs to confirm HA retention.

Results

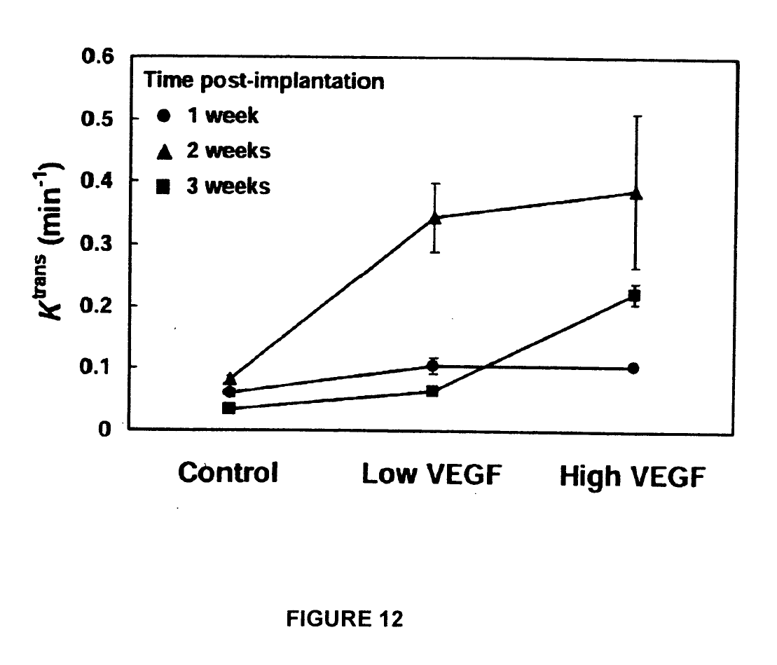

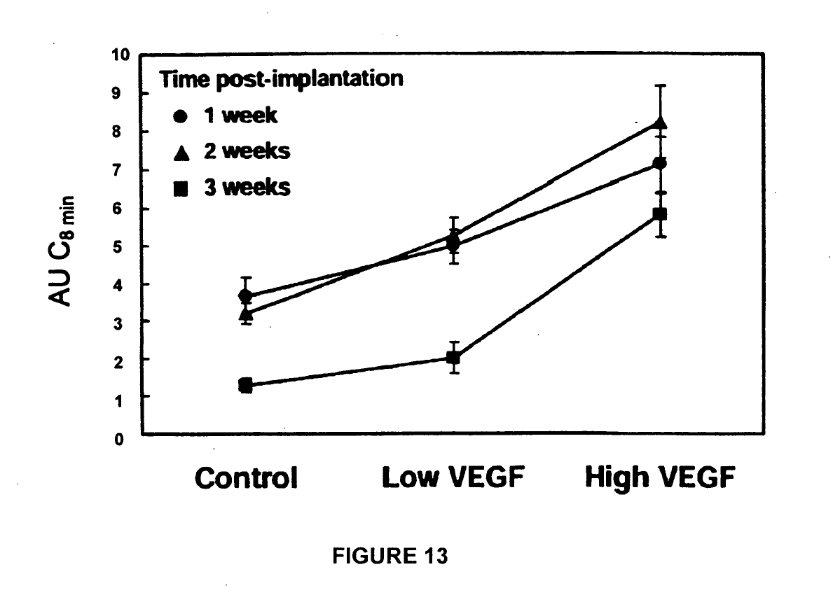

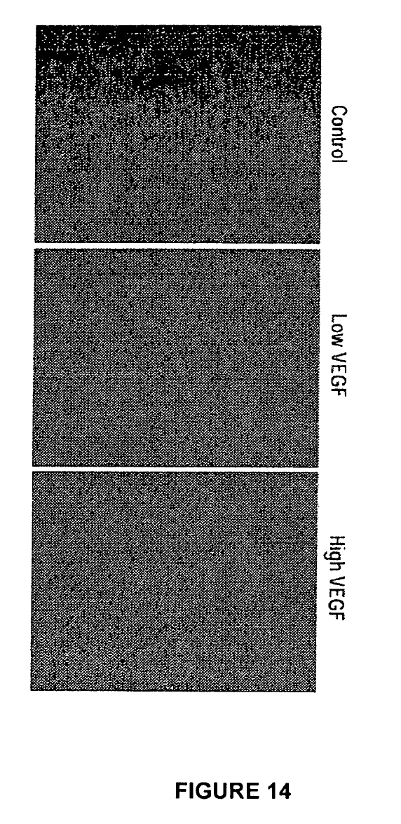

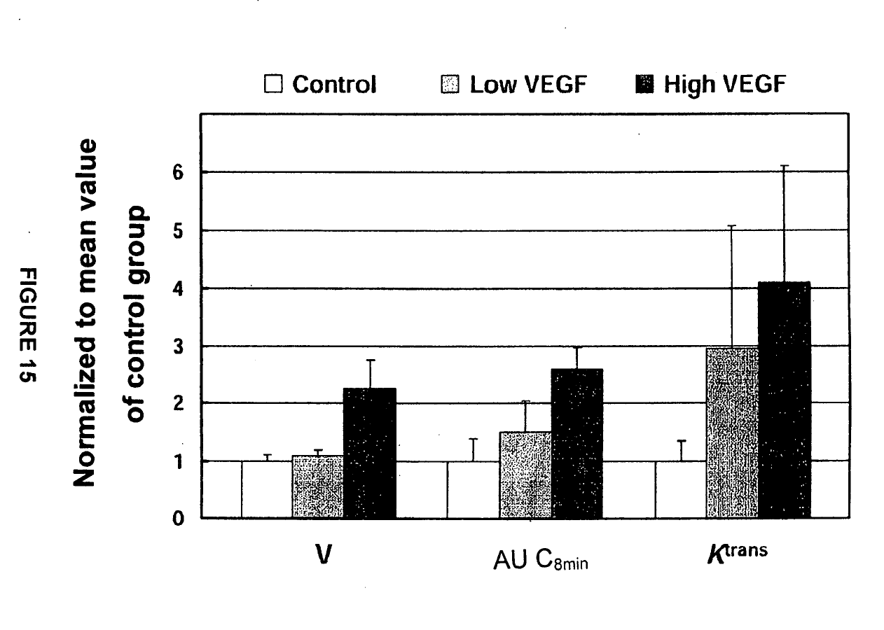

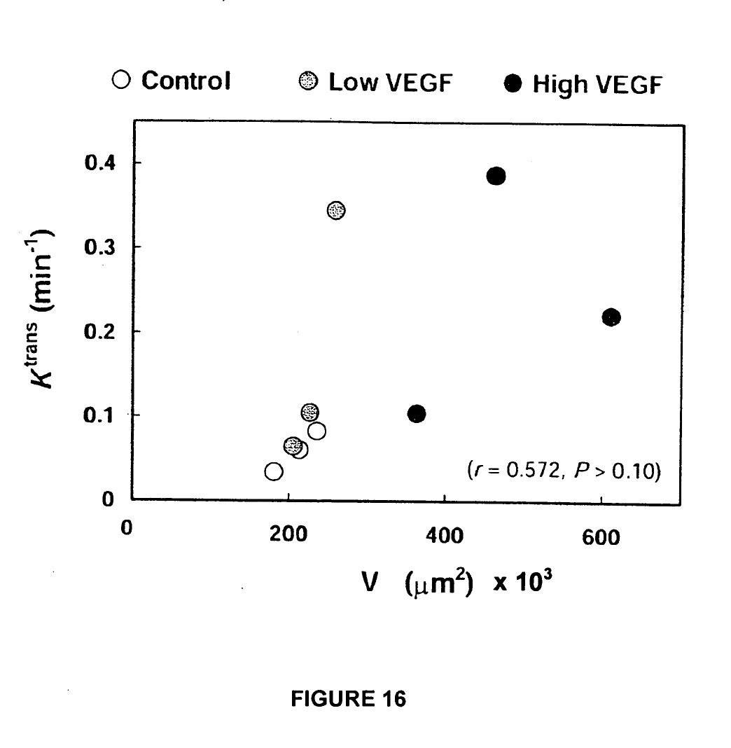

Alcian blue staining and FACE qualitatively and quantitatively confirmed HA uptake by the ACM; furthermore, Alcian blue stain confirmed the retention of HA at the end of the porosity testing (FIG. 8A, B, C). Porosity results for the three groups are summarized in FIG. 9. As the porosity values did not demonstrate a normal distribution, a nonparametric statistical analysis (Mann Whitney nonparametric test) was completed to evaluate statistical significance. Mean (±SE) porosity values were 9.8 (±1.6), 0.74 (±0.4), and 0.09 (±0.02) cc/cm2 hr for the ACM, lyophilized ACM, and HA-ACM groups, respectively (median 7.2, 0.18, 0.05) (FIG. 3B), with a statistically significant difference among all groups. (ACM vs lyophilized ACM, p<0.0001; ACM vs HA-ACM, p<0.0001; and HA-ACM vs lyophilized ACM, p=0.014).

For the 3 h testing time period in all three groups, the average volume of effluent collected in the first hour was less than that during each of the subsequent 2 h. This increase was statistically significant between the first and third hour in all groups (Table 2); however, there was no statistically significant difference between the mean effluent volumes for the second and third hour in any of the groups.

TABLE 2
Average Hourly Effluent (cc) by Group and Students 2-Tailed t-Test
Values Comparing Different Hours Demonstrating a Statistically
Significant Difference Between the First and Third, but not the
Second and Third Hours

| | | | | Student 2-Tailed | | |
|---|---|---|---|---|---|---|
| | | | | Paired t-Test (p Values) | | |
| Average Hourly | | | | Hour 1 | Hour 1 | |
| Effluent Volume (cc) | | | | vs | vs | Hour 2 vs |
| | Hour 1 | Hour 2 | Hour 3 | Hour 2 | Hour 3 | Hour 3 |
| ACM | 28.51 | 31.20 | 32.35 | 0.071 | 0.043 | 0.080396 |
| Lyophilized | 2.14 | 2.45 | 2.42 | 0.025 | 0.02 | 0.280865 |
| ACM | | | | | | |
| HA-ACM | 0.20 | 0.29 | 0.35 | 0.043 | 0.004 | 0.364885 |

Values in italic indicate p < 0.05.

### EXAMPLE 3

### Experimental Model for Testing Bladder Augmentation

The domestic pig bladder offers the closest approximation to the human bladder in terms of size, shape, tissue histology and compliance, furthermore, being a growing animal model, pig would be comparable to children as a suitable model. Operative procedures on pigs resemble those performed on humans with respect to instruments and suture materials. In addition, we have developed protocols for isolation of porcine bladder urothelial, smooth muscle, and endothelial cells. Furthermore we have phenotypically characterised these cell types respectively, cytokeratin 7 for urothelium, smooth muscle actin for smooth muscle cells, and factor VIII for endothelial cells.

Protocol for Urinary bladder acellularization: Pigs averaging 20-28 Kg can be used for bladder M-ACM processing and implantation. Whole porcine urinary bladders are cut in half and extracted in detergent containing solutions as described above. Tissue samples from the acellularized porcine bladders can be taken for histology staining with H&E and with DAPI immunofluorescence to confirm acellularity.

Preparation of VEGF-HA/M-ACM constructs: Over a 24-hour period, M-ACM is lyophylized and then rehydrated in increasing concentrations of HA (0.05, 0.1, 0.2, and 0.5). Prior to VEGF (VEGF121 Sigma-Aldrich Co) incorporation, HA/M-ACM is first dehydrated in alcohol and weighed and then rehydrated in different VEGF solutions (minimum of 1 ng, 5 ng, and a maximum of 10 ng per gram of tissue). To confirm uptake of VEGF into the HA/M-ACM, cryosections can be immunostained using immunofluorescence labelling.

Animal preparation: Nude mice can be used for this aim, since they accept xenografts and permit evaluation of angiogenesis without complications of immune rejection. All animal protocols are conducted in accordance with the public health service policy on humane care and use of laboratory animals (CGAC guidelines). Mice are anaesthetized with Ketamine (intramuscular) and pentobarbital (intraperitoneally). Sterile techniques are used to implant intra-peritoneal 1 piece (2×2 cm) of the 4 different type of the acellular matrices [HA/M-ACM, VEGF-HA/M-ACM 1 ng, and VEGF-HA/M-ACM 5 ng VEGF-HA/M-ACM 10 ng]. Implants are marked with non-absorbable sutures, left for a 1-week period and then removed for histology. Following harvesting at the designated time point, size measurement of the implanted matrix can be done prior to fixation in formalin for 24 hours, and compared to the original size at the time of implantation. Specimens can then be processed for routine histology (H&E) and immunohistochemistry staining for endothelial cells (Factor VIII). A pathologist blinded to the groups and the hematoxylin and eosin (H&E) stained paraffin embedded sections can score factor VIII immunohistochemical stain to obtain a vascular index. Particular attention should be paid to the periphery and centre of the graft, and relative numbers of blood vessels present per sample can provide a measure of the extent of angiogenesis. For each slide, 5 random images can be captured using 20× objective; angiogenesis is expressed as percent blood vessels, relative to the total area of tissue in each image. It is estimated that from each sample, an average of 3 slides can be processed. To achieve the objectives of the study and using an alpha value of 0.05 and a power of 0.95 the total number of animals needed is estimated to be 20 (5 for each arm).

### EXAMPLE 4

### Dynamic Contrast Enhanced MRI for Measuring Neovascularization in Tissue-Engineered Organ Constructs

Materials and Methods

Bladder Graft Preparation and Animals

Nine female New Zealand white rabbits (3.0-3.5 kg) were used to evaluate the role of DCE-MRI to track neovascularization over time in tissue-engineered urinary bladder constructs fortified with different levels of vascular endothelial growth factor (VEGF). Whole rabbit urinary bladders were acellularized using a proprietary chemical method (U.S. Pat. No. 4,776,853 incorporated herein by rerference). Over a 24-hour period, the acellular matrix (ACM) was dry-frozen and then rehydrated in a hyaluronic acid (HA) solution containing different concentrations of VEGF (0, 10, and 20 ng per gram of tissue). Implantation of the composite hybrid (ACM HA) was performed intra-peritoneally onto the anterior bladder wall of three rabbits for each VEGF level. At different time points post-implantation (1, 2, and 3 weeks), one rabbit from each VEGF group underwent MRI. Anesthesia was induced by a mixture of ketamine, rompun, and atropine (18, 4, and 0.02 mg/kg bodyweight, respectively) injected intramuscularly, and maintained during MRI with pentobarbital (6-13 mg/kg) administered through a 22-gauge needle inserted in the ear vein. After MRI, animals were euthanized by an overdose of pentobarbital, and the bladders were subsequently removed for immunohistochemical staining. All experimental protocols were approved by the Animal Care Committee of the Hospital for Sick Children and were in compliance with the Canadian Council for Animal Care Guidelines.

MRI

Imaging was performed on a Signa LX whole-body MRI scanner (General Electric Medical Systems, Milwaukee, Wis.) operating at 1.5 Tesla. Animals were placed in a prone position and imaged with a cardiac phased array coil to improve signal uniformity over the field of view.

Identification of the bladder constructs was achieved using T₂-weighted multislice fast spin-echo (FSE) images with the following parameters: TR/TE=4000/105 msec, 8 ETL, 2 NEX, matrix=256×192, slice thickness (SL)=3 mm, field of view (FOV)=12 cm. Dynamic images were then acquired using a 3D T₁-weighted fast spoiled gradient recalled echo (SPGR) sequence: TR/TE=9.3/2.1 msec, flip angle (FA)=15°, bandwidth (BW)=15.62 kHz, FOV=12 cm, matrix=256×192×12, SL=3 mm, 1 NEX. These images were acquired prior to, during, and for approximately 8.5 minutes following rapid bolus injection of Gd-DTPA (Magnevist, Berlex Canada, Montreal), injected intravenously at a dose of 0.1 mmol/kg. Each 3D data set required approximately 28-seconds acquisition time. Pre-injection baseline T₁-maps were acquired using the Look-Locker (Look et al. Rev Sci Instrum 1970; 41:250-251; Kay et al. Magn Reson Med 1991; 22:414-424, incorporated herein by reference) method (TR/TE=3000/225 msec, 1 echo, FA=20°, BW=125 kHz, FOV=12 cm, SL=3 mm, 4 NEX). High resolution, fat-suppressed T₁-weighted FSE images were also acquired before and following contrast administration (TR/TE=500/15 msec, 4 ETL, 3 NEX, matrix=256×192, SL=3 mm, FOV=12 cm).

Analysis of MRI Data

The location of the grafted ACM construct was identified in each rabbit on sagittal and axial T₂-weighted FSE images. Correct identification of the ACM on MRI was confirmed by observations of its shape and location on the bladder, and comparing them to photographs taken of the bladder upon harvest. Contrast enhancement characteristics were determined for four or five contiguous 3-mm image slices through the central portion of the ACM construct. On each imaging slice, a region-of-interest (ROI) was outlined to include the most uniformly enhancing area on T₁-weighted images 4 minutes post-injection. Fatty inclusions were excluded from the ROI. The average signal intensity was determined in the ROI and used to calculate the post-contrast T₁, based on knowledge of the pre-contrast T₁ value and the signal intensity equation for a FSPGR sequence (Cheng et al. J Magn Reson Imaging 2003; 18:585-598; Cheng et al. J Magn Reson Imaging 2004; 19:329-341, incorporated herein by reference). The tracer concentration was then estimated assuming a linear relationship with the change in 1/T₁ (Tofts et al. J Magn Reson Imaging 1997; 7:91-101, incorporated herein by reference). Pharmacokinetic analysis of tracer uptake was performed in the ROI on each slice, and the results were averaged over all slices for each animal.

Several analysis methods for DCE-MRI were considered to determine which method, if any, allowed discrimination of grafts prepared with different levels of VEGF in a blinded animal study, and also which correlated best with histological measures of microvessel density.

Enhancement Rate (SSₘₐₓ)

The SSₘₐₓ was computed as the maximum slope on the concentration-time curve based on the two successive points that yielded the largest change in concentration. This approach is a modification of techniques described previously (Buadu et al. Radiology 1996; 200:639-649; Flickinger et al. Magn Reson Imaging 1993; 11:617-620, incorporated herein by reference) for characterizing the signal-intensity, rather than the concentration, profile. Another commonly used index, the time-to-peak (Stack et al. Radiology 1990; 174:491-494, incorporated herein by reference), was not considered due to continued enhancement observed in a number of cases.

Tofts and Kermode Model (K^{trans}, vₑ, vₚ)

This two-compartment, bi-directional model describes the transport of contrast medium across the endothelium (Tofts et al. J. Magn Reson Imaging 1997; 7:91-101, incorporated herein by reference). Three physiological parameters were estimated: the endothelial transfer constant (K^{trans}), fractional plasma volume (vₚ), and extravascular extracellular volume fraction (vₑ). The extended model was implemented assuming biexponential decay of the AIF using Tofts' data for rabbits (Tofts et al. Magn Reson Med 1991; 17:357-367, incorporated herein by reference). Nonlinear least-squares fitting of Tofts model to the concentration-time curve was performed to estimate K^{trans}, vₑ, and vₚ.

Uptake Integral Approach (AUC_{1 min}, AUC_{2 min}, AUC_{8 min})

The initial area-under-the-concentration-time curve (AUC) has been suggested by Evelhoch (20) to be equivalent to Tofts' K^{trans} when the first 60-90 seconds post-injection is considered.

The impact of variations in arterial input is minimized by normalizing the AUC values to that in a reference tissue. In our implementation, the concentration-time curve was integrated over time intervals of 1, 2, and 8 minutes post-injection and normalized to resting dorsal muscle.

Therefore, a total of seven DCE-MRI parameters were determined for each region.

Immunohistochemistry

After sacrificing the rabbits, a 2×2cm² area of the bladder wall, including the central portion of the ACM and normal bladder, was harvested. The specimens were whole-mount immunostained using the anti-CD31 antibody clone JC/70(Dako) and then scanned using confocal laser scanning microscopy (×10 Carl Zeiss Axiovert 200) to search for the central ACM area, where the microvessel count was highest. Thirty layers were scanned from this region and merged using LSM 510 software. The microvascular area (μm²) was then measured from the composite image using SimplePCI software to yield an estimate of the microvessel density (MVD).

Statistical Analysis

A two-way analysis of variance (ANOVA) was performed to determine whether MRI-derived parameters (SSₘₐₓ, K^{trans}, vₚ, vₑ, AUC_{1 min}, AUC_{2 min}, AUC_{8 min}) and histological counts of microvessel density (MVD) varied significantly with VEGF concentration. Post-hoc analysis of differences amongst means was based on Tukey-Kramer HSD method. The ability of MRI to identify different VEGF levels at each post-implantation interval was also tested; precision was assessed with a two-tailed Student's t-test. Finally, MRI parameters were compared to MVD using Spearman rank correlation, and linearity was tested by Pearson correlation analysis.

Results

DCE-MRI Results

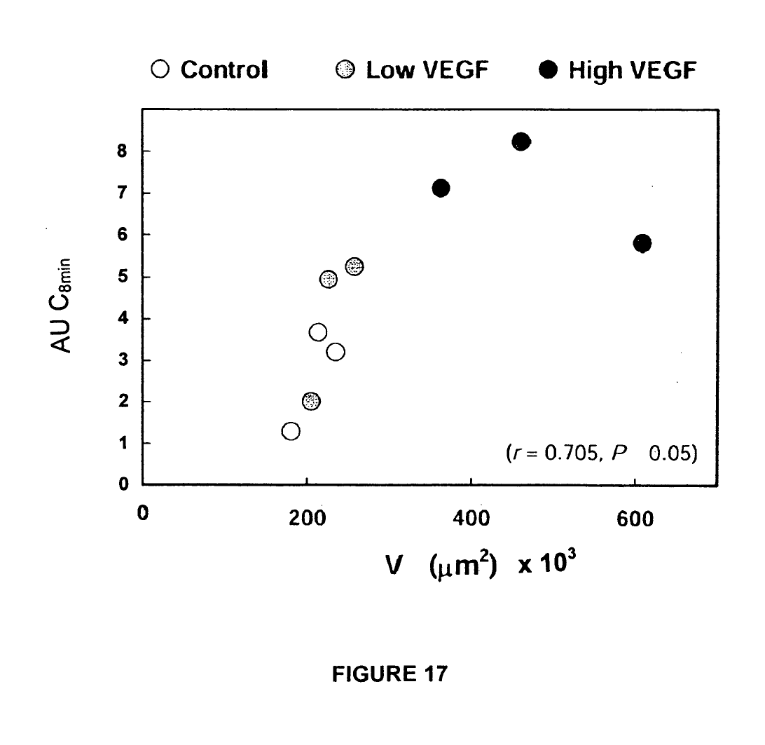

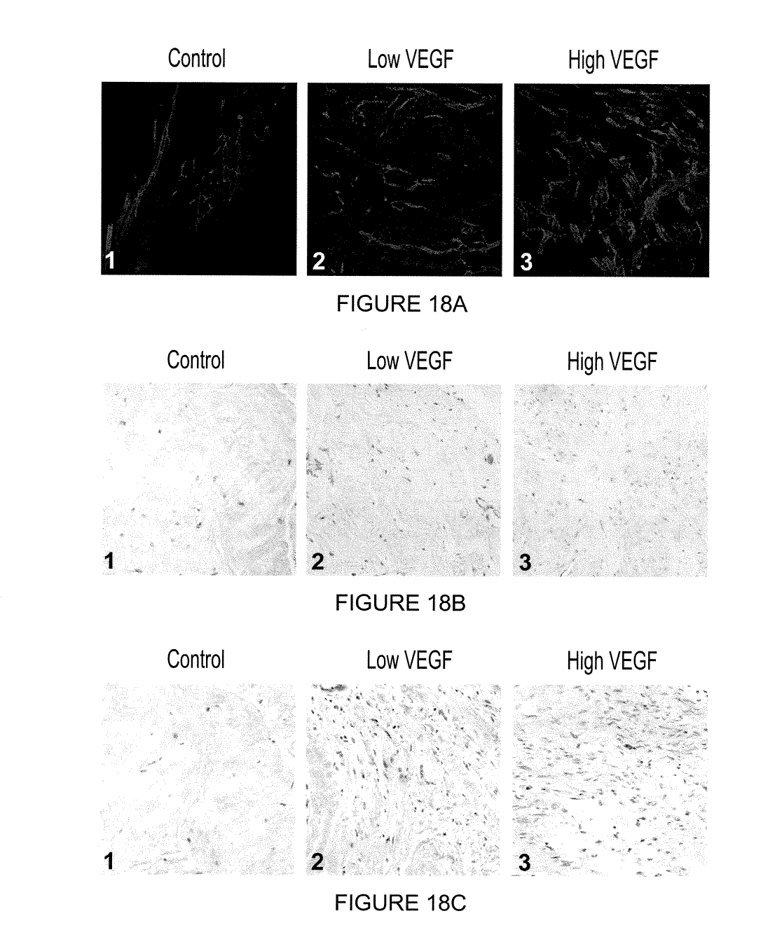

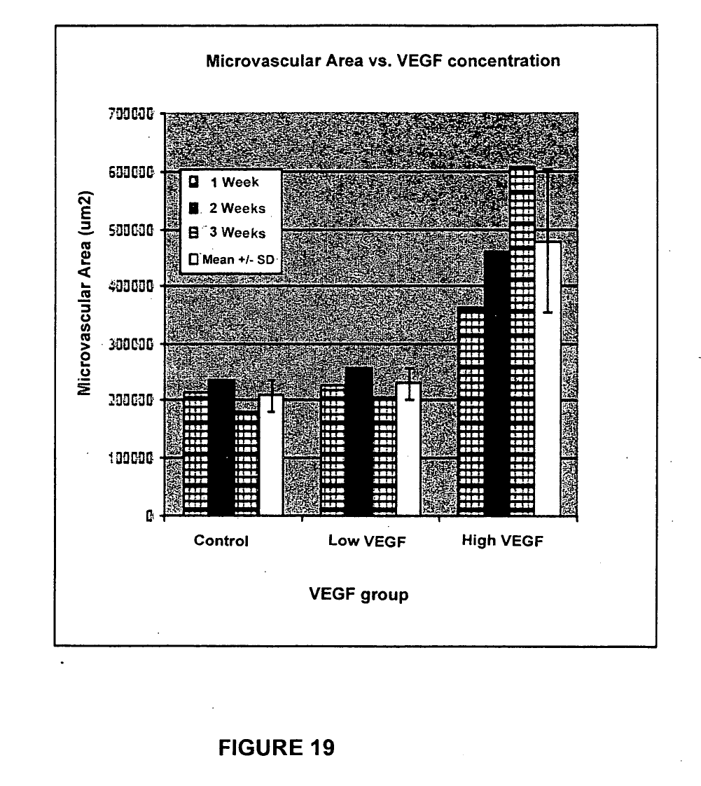

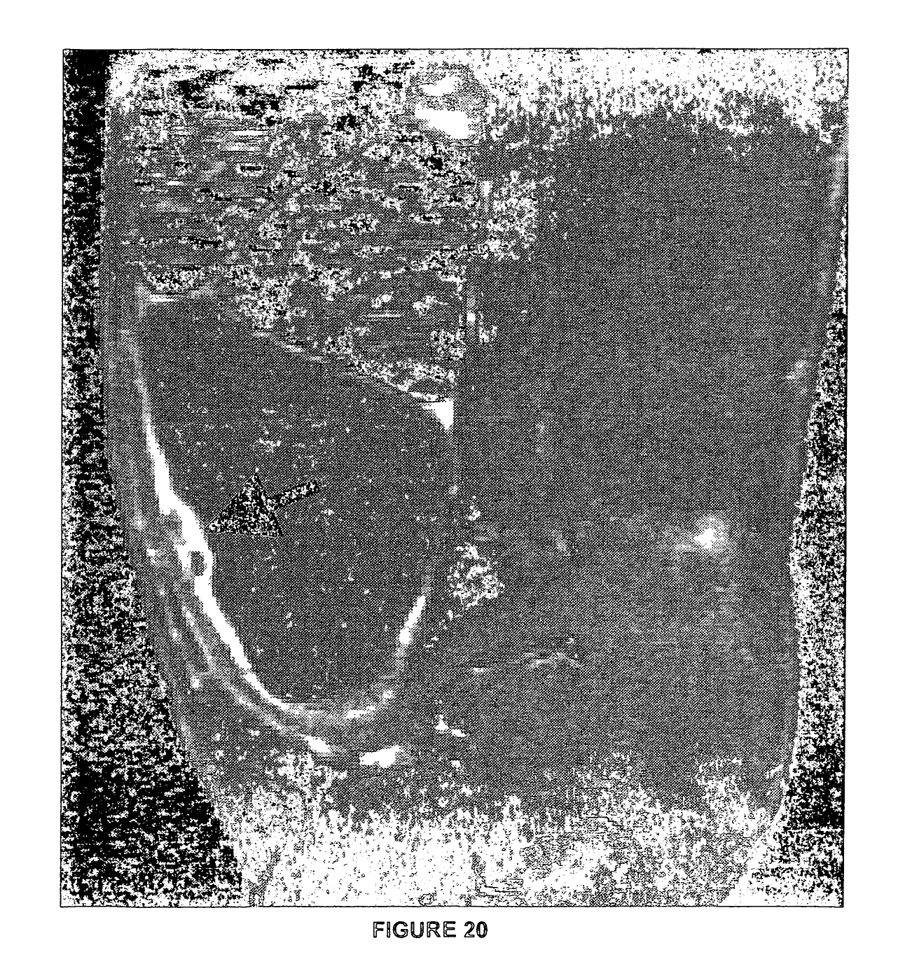

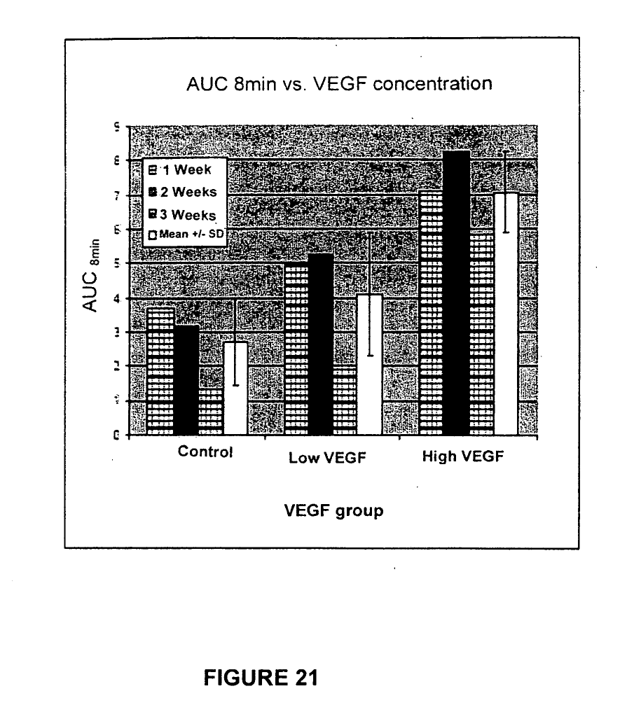

Representative contrast-enhanced images are shown for grafts prepared with three different levels of VEGF at 1 week post-implantation (FIG. 10). The corresponding concentration-time curve for each animal is shown in FIG. 11 and represents an average across at least four contiguous image slices. In all cases, the graft was identified by a thickening at the site of implantation and greater enhancement relative to surrounding bladder tissue. Non-enhancing regions within the mass were present in some cases and were excluded from analysis. In general, faster and greater contrast uptake was obtained with increased concentrations of VEGF. To assess the accuracy and precision of DCE-MRI in discriminating different preparations of VEGF in the same post-implantation interval, parameter values were ranked in increasing order and differences between animals were tested for statistical significance. FIGS. 12 and 13 illustrate the mean value (±SE) for the parameters K trans and AUC 8 min, respectively, in all animals. Both parameters correctly distinguished all VEGF groups, on the assumption of increased values with higher VEGF levels. The control and low VEGF groups were best distinguished with K trans (P≦0.011, two-tailed t-test). However, the low and high VEGF groups were best distinguished with AUC8 min, especially in the first two weeks post-implantation, where a significant increase was seen with AUC8 min (P<0.025) but not with K trans (P>0.37). Table 3 summarizes the discrimination power of all DCE-MRI parameters. Cases where the assumption of a positive relationship between MRI and VEGF resulted in an incorrect classification are labeled “n.a”. Of all the parameters, only SSmax, K trans, AUC 2 min, and AUC8 min correctly classified all cases. The parameter AUC8 min was the most precise (small uncertainties (P<0.05) were consistent in all but one case—Table 2) and, therefore, had the best discrimination power. CD31 Microvessel Density and VEGF CD31 microvessel density (MVD) counts were obtained for all animals. The appearance of microvessels in grafts examined 1 week post-implantation is shown in the immunostained composite images of FIG. 14. At all times post-implantation, higher MVD was seen with increased levels of VEGF. However, the change in mean MVD was minimal (P=0.96, postANOVA) between the control and low VEGF groups (mean±SD: 210497±22306 μm 2 versus 230187±21604 μm 2) (see Table 4 and FIG. 15). In contrast, a significant increase (P=0.045) was observed in the high VEGF (477894±101200 μm 2) compared to the low VEGF group. DCE-MRI versus Microvessel Density Changes in microvessel density (MVD) and DCE-MRI parameters at varying levels of VEGF are summarized in Table 4, where time-related tissue changes are averaged across the different intervals following implantation. Notice the small variability in MVD in the control and low VEGF groups, which suggests the influence of time-related changes is not significant at these concentrations. The small increase in MVD (˜9%) in the low VEGF group compared to control was accompanied by much larger changes in MRI parameters. K trans and SS max exhibited the greatest increase (˜3-folds for both), whereas AUC8 min exhibited the smallest change (1.5-folds). However, for the large difference in MVD that was observed between the low and high VEGF groups (over 2-folds increase, P=0.045), relative increases in MRI parameters were much smaller than expected, ranging from 1.3 to 1.7. Compared to other MRI indices, AUC estimates showed the largest relative increase and smallest variability. In fact, AUC8 min was the only MRI parameter that demonstrated a significant difference between the low and high VEGF groups (P=0.0058). In FIG. 15, the relative changes and variability of two MRI parameters, K trans and AUC8 min, are compared to highlight their contrasting behavior at different VEGF levels and to illustrate their association to changes in MVD. Linear regression was performed to evaluate the relationship between DCE-MRI and MVD. Spearman rank correlation analysis showed that MRI parameters K trans, SS max, AUC2 min, and AUC8 min were comparable in their ability to correctly rank data according to vessel density (ps=0.88-0.92, P<0.05). Plasma volume estimates were poorest with Tofts' vp (ps=0.73, P<0.05). Despite the positive association between these MRI parameters and MVD, the relationships were not strictly linear. Pearson's correlation analysis showed that K trans was poorly correlated to MVD (r=0.572, P=0.11), as illustrated in the scatterplot of FIG. 16. The poor correlation was due mainly to large differences in vessel density between the low and high VEGF groups with relatively minor changes in K trans. Significantly greater correlation was achieved with the uptake integral method, with improved linearity between AUC and MVD for shorter time integrations (r=0.705-0.817, P≦0.034). The high degree of correlation to MVD is demonstrated in FIG. 17 for AUC8 min, the only MRI parameter to identify correctly the VEGF dosage in each postimplantation time interval and to show a significant change concordant with the increased vessel density in the high VEGF group.

TABLE 3
Precision of DCE-MRI Parameters for Discriminating Different VEGF
Preparations at Various Post-Implantation Intervals*.

| | 1 week | | 2 weeks | | 3 weeks | |
|---|---|---|---|---|---|---|
| | post-implantation | | post-implantation | | post-implantation | |
| | Control vs. | Low vs. | Control vs. | Low vs. | Control vs. | Low vs. |
| | Low VEGF | High VEGF | Low VEGF | High VEGF | Low VEGF | High VEGF |
| SSₘₐₓ (mM/min) | 0.012 | 0.44 | 0.002 | 0.43 | <0.001 | <0.001 |
| K^{trans} (min⁻¹) | 0.011 | 0.50 | 0.002 | 0.37 | <0.001 | <0.001 |
| νₚ | 0.078 | n.a. | 0.007 | n.a. | <0.001 | <0.001 |
| νₑ | 0.22 | 0.15 | 0.018 | n.a. | <0.001 | 0.008 |
| AUC | 0.44 | n.a. | <0.001 | 0.070 | 0.061 | <0.001 |
| AUC | 0.16 | 0.38 | <0.001 | 0.13 | 0.033 | <0.001 |
| AUC | 0.049 | 0.024 | 0.005 | 0.010 | 0.076 | <0.001 |

*Shown are P-values from two-tailed Student's t-tests for pair-wise comparisons of means.
Significant differences (P < 0.05) are indicated in bold.
Incorrect classification by MRI parameters is indicated by n.a.

TABLE 4
Comparison of CD3i Microvessel Density (MVD) and DCE-MRI
Parameters at Different VEGF Concentrations*.

| | | | | P-value | | |
|---|---|---|---|---|---|---|
| | | | | Control | Control | Low |
| | | | | vs. Low | vs. High | vs. High |
| | Control VEGF | Low VEGF | High VEGF | VEGF | VEGF | VEGF |
| MVD (μm²) | 210497 ± 22306 | 230187 ± 21604 | 477894 ± 101200 | 0.96 | 0.035 | 0.045 |
| SSₘₐₓ (mM/min) | 0.038 ± 0.013 | 0.108 ± 0.079 | 0.145 ± 0.068 | 0.31 | 0.12 | 0.67 |
| K^{trans} (min⁻¹) | 0.058 ± 0.020 | 0.171 ± 0.124 | 0.238 ± 0.116 | 0.32 | 0.12 | 0.62 |
| νₚ | 0.0155 ± 0.0127 | 0.0384 ± 0.0141 | 0.0491 ± 0.0171 | 0.48 | 0.27 | 0.84 |
| νₑ | 0.328 ± 0.104 | 0.500 ± 0.203 | 0.474 ± 0.050 | 0.33 | 0.42 | 0.97 |
| AUC | 1.71 ± 1.21 | 3.19 ± 1.56 | 5.43 ± 1.91 | 0.72 | 0.22 | 0.50 |
| AUC | 1.85 ± 1.05 | 3.30 ± 1.56 | 5.34 ± 1.17 | 0.56 | 0.12 | 0.36 |
| AUC | 2.71 ± 1.04 | 4.07 ± 1.47 | 7.04 ± 0.99 | 0.08 | 0.0014 | 0.0058 |

*Values are reported as mean ± S.D. P-values are calculated from Tukey-Kramer HSD test for pair-wise comparisons following two-way ANOVA analysis.
Significant differences (P < 0.05) are indicated in bold.

### EXAMPLE 5

### Materials and Methods

ACM-HA VEGF Constructs:

Whole rabbit urinary bladders were acellularized using a proprietary chemical method. H&E staining and DAPI immunoflourescence confirmed acellularity. Over 24-hours ACM was lyophilized and then rehydrated in increasing concentrations of hyaluronic acid (0.05, 0.1, 0.2, and 0.5 mg/100 ml). The HA-ACM construct was dehydrated in alcohol, lyophilized and then rehydrated in one of three different VEGF concentrations (0 ng, 10 ng, or 20 ng/gram ACM). Constructs were maintained in VEGF solution overnight prior to implantation. HA incorporation was confirmed with Alcian blue staining.

Graft Implantation:

Nine female New Zeland white rabbits (3.0-3.5 kg) were grouped (n=3 per group) for the three different VEGF concentrations. Animals were fasted overnight. Anesthesia was induced with Akmezine followed by endotracheal intubation, automatic ventilatory support, and maintenance anesthesia with a combination of 1.5% Halothane and oxygen. Antibiotic prophylaxis was administered (I.V. Penicillin) on induction. ACM-HA VEGF constructs (n=3 for each VEGF concentration, 4×4−5×5 cm²) were implanted extraperitoneally onto the anterior bladder wall through a lower midline incision with non-absorbable suture for graft identification at harvest. The abdomen was closed in two layers. Animals were recovered in the animal facility recovery room and returned to regular animal pens. Animals received antibiotics (Penicillin and Streptomycin) and analgesia (Tamgesic I.M.) for one week postoperatively.

At 1, 2 and 3 weeks one rabbit from each VEGF group was anesthetized with intramuscular injection of ketamine, rompun and atropine (14, 4, and 0.02 mg/kg body weight respectively) with pentobarbital maintenance (6-13 mg/kg) and DCE-MRI was completed. After MRI, animals were euthanized with pentobarbital overdose and bladders were harvested.

All experimental protocols were approved by the Animal Care Committee of the Hospital for Sick Children and in compliance with the Canadian Council for Animal Care guidelines.

MRI:

MRI was completed on a 1.5-Tesla Signa LX whole-body MRI scanner (General Electric Systems, Milwaukee, Wis.) using a cardiac phased array coil with the animal in the prone position. The radiologist was blinded to the VEGF concentrations.

Bladder constructs were identified utilizing T₂-weighted multi-slice fast spin echo (FSE) images. Dynamic images were acquired using a 3D T₁-weighted fast spoiled gradient recalled echo (FSPGR) sequence prior to, during, and for 8.5 minutes after rapid intravenous bolus of 0.1 mmol/kg Gd-DTPA (Magnevist, Berlex Canada, Montreal). Baseline T₁ maps were obtained prior to injection and high-resolution fat-suppressed T₁-weighted FSE images were acquired before and after contrast administration.

MRI spatial resolution was 0.47×0.63 mm. Grafted ACM was localized in each rabbit on sagittal and axial T₂-weighted FSE images. Correct identification of the ACM on MRI was confirmed by comparison to bladder harvest photographs. On each imaging slice a region of interest (ROI) was outlined to include the most uniformly enhancing area on T₁-weighted images 4 minutes post injection, excluding fatty inclusions. Average ROI signal intensity was used to calculate the post-contrast T₁, based on the pre-contrast value and the signal intensity equation for a FSPGR sequence. Tracer concentration was estimated assuming a linear relationship with the change in 1/T₁. Pharmacokinetic analysis of tracer uptake was performed in the ROI for four contiguous 3 mm slices through the ACM center, and results were averaged over all slices. Area under the concentration-time curve (AUC) was integrated for the ROI at 1, 2, and 8 minutes after injection and normalized to resting dorsal muscle.

Immunohistochemistry:

A 2×2 cm² segment of the bladder wall, including the central portion of the ACM-hybrid and normal bladder, was harvested at rabbit sacrifice. Microdissection separated ACM from native bladder. Cryosections were washed in dimethyl sulfoxide and PBS and then incubated in PBS containing 15% goat serum for 1 hour at room temperature to block non-specific binding. ACM grafts were stained with monoclonal antibody to CD31 clone JC/70(DAKO), a marker for vascular endothelium, at a 1:30 dilution and incubated overnight in a humidified chamber at 4° C. Sections were washed with PBS and incubated at room temperature for 2 hours with flourochrome-labeled secondary antibody, chicken anti-mouse Alexa 594 (Molecular Probe) at 1:200 dilution. After washing again in PBS, sections were mounted in 90% glycerol/PBS containing p-phenylethylenediamine (1 mg/ml) to reduce flurochrome bleaching.

Fluorescent images of microvascular whole mount staining were obtained using a confocal laser scanning microscope (×10 Carl Zeiss Axiovert 200) with LSM 510 program software (version 3.2), coupled to a color charge-coupled device (CCD) video camera, a digitizing card and a monitor. Thirty layers were scanned to measure CD31 staining from the composite image using Simple PCI software to estimate microvascular area (MVA) (μm²).

Histology:

Central sections were stained with Masson trichrome and H&E for cellularity and fibrosis.

Statistical Analysis:

Statistical analyses were performed using a two-tailed Student's t-test to determine whether MRI AUC_{1 min, 2 min, 8 min} and histological MVA's were significantly different for varying concentrations of VEGF over time, and whether MRI could correctly predict the VEGF dose. MRI AUC and MVA were compared with Spearman rank correlation and Pearson's linear regression.

Results:

Microvascularity and VEGF:

Microvascular staining is shown in FIG. 18A. At all times, higher MVA was seen with increasing levels of VEGF. Change in mean MVA was minimal between control and low VEGF groups and did not reach statistical significance (mean±SD: 210,497±27,319 μm² vs. 230,187±26,460 μm²; P=0.21). However, compared to the low VEGF group, the high VEGF group (20 ng/g) showed a significant increase in mean MVA (477,894±123,945 μm², P=0.014). (FIG. 19) Additionally, MVA variability between control and low VEGF groups was small, suggesting that time related changes may not be a significant influence at these VEGF levels.

Histology:

Good biocompatibility was manifested by cellular repopulation, little inflammatory response, and similar neovascularity at the implant edge compared to the center. Vascularization was accentuated with higher VEGF levels. At one week grafts from all groups demonstrated moderate cellularity and fibrosis. However, by the third week, the high VEGF graft maintained moderate degrees of cellularity and fibrosis, while grafts from the control and low VEGF groups had low cellularity and dense fibrosis. (FIG. 18B)

DCE-MRI:

In all cases, grafts were identified by a thickening at the implantation site and greater enhancement relative to surrounding bladder (FIG. 20). Non-enhancing regions were present in some cases and excluded from analysis. Generally, faster and greater contrast uptake was obtained with increased concentrations of VEGF.

To assess the ability of DCE-MRI to discriminate grafts prepared with varying VEGF levels, AUC differences among animals at the same time post-implantation were tested for statistical significance. AUC correctly distinguished all VEGF groups, assuming that increased values corresponded to higher VEGF levels. AUC's _{8 min}, representing an average across four contiguous image slices are depicted in FIG. 21 as a function of VEGF. A significant increase was noted in AUC₈ min at all times after implantation between low and high (10 and 20 ng) VEGF groups (P<0.025). Only AUC_{1 min} at one week failed to correctly classify VEGF concentration (low vs. high).

DCE-MRI versus MVA and VEGF:

The relationship between MRI AUC and MVA was evaluated first by comparing mean values within VEGF groups. MVA difference between control and low VEGF groups was modest (P=0.21) as were all AUC differences at these levels (P˜0.17). A relatively small MVA change (˜9%) between control and low VEGF groups was accompanied by a much larger AUC increase (50% AUC_{8 min}). Comparing low and high VEGF groups, where MVA difference was over two-fold (P=0.01), a statistically significant, but smaller relative difference was also observed in AUC₈ min (P=0.038). Vascular changes from VEGF other than MVA may explain the significant change in AUC without significant MVA increases at low VEGF concentrations.

Linear regression analysis showed that all AUCs correlated with MVA (Pearson's correlation coefficient r=0.71, 0.79, 0.82 for AUC_{8 min,} AUC_{2 min}, AUC_{8 min}). Furthermore, all AUCs correctly ranked the data, except AUC_{1 min} for one rabbit. (FIG. 22).

While the invention has been described in connection with specific embodiments thereof, it will be understood that it is capable of further modifications and this application is intended to cover any variations, uses, or adaptations of the invention following, in general, the principles of the invention and including such departures from the present disclosures as come within known or customary practice within the art to which the invention pertains and as may be applied to the essential features herein before set forth, and as follows in the scope of the appended claims.

1. A scaffold for implant in a mammal comprising:
a) an acellular matrix;
b) one or more biocompatible polymer; and
c) one or more biomimetic agent.

2. The scaffold as claimed in claim 1 wherein the acellular matrix is derived from a biological tissue.

3. The scaffold as claimed in claim 2 wherein the acellular matrix comprises extracellular matrix proteins.

4. The scaffold as claimed in claimed in claim 3 wherein the extracellular matrix proteins are selected from laminin, fibronectin, collagen IV, collagen I, collagen III, desmin, smooth muscle actin, smooth muscle myosin, vimentin, PAN neurofilament and combinations thereof.

5. The scaffold as claimed in claim 1 wherein the biocompatible polymer is a polysaccharide.

6. The scaffold as claimed in claim 5 wherein the polysaccharide is a glycan.

7. The scaffold as claimed in claim 6 wherein the glycosaminoglycan is hyaluronic acid.

8. The scaffold as claimed in claim 1 wherein the biomimetic agent is selected from angiogenic agent, hormone, protein, cytokine, epidermal growth factor and nerve growth factor.

9. The scaffold as claimed in claim 8 wherein the biomimetic agent is an angiogenic agent.

10. The scaffold as claimed in claim 9 wherein the angiogenic agent is VEGF.

11. The scaffold as claimed in claim 1 further comprising cells compatible with a target organ for implant.

12. A biological tissue comprising a scaffold as claimed in claim 1.

13. A scaffold for implant in a mammal comprising:
a) an acellular matrix comprising substantially intact extracellular matrix proteins: and
b) a biocompatible polymer.

14. The scaffold as claimed in claim 13 wherein the acellular matrix is derived from a biological tissue.

15. The scaffold as claimed in claimed in claim 14 wherein the extracellular matrix proteins are selected from laminin, fibronectin, collagen IV, collagen I, collagen III, desmin, smooth muscle actin, smooth muscle myosin, vimentin, PAN neurofilament and combination thereof.

16. The scaffold as claimed in 13 wherein the biocompatible polymer is a polysaccharide.

17. The scaffold as claimed in claim 16 wherein the polysaccharide is a glycan.

18. The scaffold as claimed in claim 17 wherein the glycosaminoglycan is hyaluronic acid.

19. A biological tissue comprising a scaffold as claimed in claim 13.

20. A method for preparing an acellular matrix comprising:
a) obtaining a tissue sample from a biological tissue;
b) treating the tissue to remove cells from the tissue sample with the proviso that the treatment does not comprise extraction of the tissue sample with an anionic detergent.

21. The method as claimed in claim 20 wherein the step of treating comprises removing cells by lysing cells, digesting nucleic acids and extracting the tissue.

22. The method as claimed in claim 21 wherein the steps of lysing, digesting and extracting comprise:
a) treating the tissue sample with a hypotonic buffer solution at a mild alkaline pH for rupturing cells of the tissue sample, the hypotonic buffer solution including active amounts of proteolytic inhibitors and active amounts of antibiotic;
b) extracting the tissue sample obtained in step a) with a buffered solution having a high concentration of salt, the solution being at a mild alkaline pH and including a non-ionic detergent;
c) subjecting the tissue sample obtained in step b) to enzymatic digestion in a buffered saline solution, the enzymes consisting of purified protease-free deoxyribonuclease and ribonuclease;
d) extracting the tissue sample obtained in step c) with a buffered solution at a mild alkaline pH and including one or more non-ionic detergents.

23. The method as claimed in claim 20 further comprising the step of incorporating a biocompatible polymer in the matrix.

24. The method as claimed in claim 23 wherein said the step of incorporating comprises:
a) freeze-drying the acellular matrix over a sufficient period of time; and
b) rehydrating the matrix with a solution comprising the biocompatible polymer.

25. The method as claimed in claim 24 wherein the biocompatible polymer is a polysaccharide.

26. The method as claimed in claim 25 wherein the polysaccharide is a glycan.

27. The method as claimed in claim 26 wherein the glycosaminoglycan is hyaluronic acid.

28. The method as claimed in claim 23 further comprising the step of incorporating a biomimetic agent.

29. The method as claimed in any one of claim 28 wherein the biomimetic agent is an angiogenic agent.

30. The method as claimed in claim 29 wherein the step of incorporating an angiogenic agent comprises rehydrating the freeze-dried matrix with a solution comprising the polysaccharide and the angiogenic agent.

31. The method as claimed in claim 30 wherein the polysaccharide is hyaluronic acid and the angiogenic agent is VEGF.

32. A method for treating a patient in need of tissue replacement/augmentation the method comprising:
a) obtaining an acellular matrix comprising a biocompatible polymer and a biomimetic agent.
b) implanting the matrix in the patient.

33. The method as claimed in claim 32 wherein the biocompatible polymer is a polysaccharide.

34. The method as claimed in claim 32 further comprising the step of monitoring the implant over time for assessing functionality.

35. The method as claimed in claim 34 wherein the monitoring is performed using magnetic resonance imaging.

36. The method as claimed in claim 35 wherein the magnetic resonance imaging is dynamic contrast-enhanced magnetic resonance imaging (DCE-MRI) the method comprising:
a) injecting a nuclear magnetic resonance contrast agent in the individual;
b) obtaining dynamic contrast-enhanced magnetic resonance imaging (DCE-MRI) measurement in a region of interest (ROI) of the tissue;
c) correlating the measurements with a parameter indicative of functionality of the tissue.

37. The method as claimed in claim 36 wherein the measurements are performed at different times after injection of the agent.

38. The method as claimed in claim 37 wherein the parameter is an integrated estimation of an area under a curve (AUC) of contrast agent concentration as a function of time.

39. The method as claimed in claim 38 wherein the AUC is correlated with microvessel density (MVD) in the tissue.

40. The scaffold as claimed in claim 4 wherein the biological tissue is bladder tissue.
